(19) United States
(12) Patent Application Publication
(10) Pub. No.: US 2012/0313006 A1
(43) Pub. Date: Dec. 13, 2012

(54) TRASH CAN WITH ULTRA-VIOLET STERILIZER

(75) Inventor: TING-CHIN CHIU, Tainan (TW)

(73) Assignee: FIRST CHANCE ENTERPRISE CO., LTD, Tainan (TW)

(21) Appl. No.: 13/156,046

(22) Filed: Jun. 8, 2011

(51) Int. Cl. A61L 2/10

(52) U.S. Cl. 250/455.11

(57) ABSTRACT

A trash can includes a can with a lid pivotably connected to the open top thereof and multiple Ultra-Violet sterilizers are connected to the underside of the lid. A sensor is connected on the top surface of the can so as to detect the position of the lid. A control unit is connected to the can and includes a power supply which is electrically connected to the Ultra-Violet sterilizers and the sensor. When the sensor detects the lid to be in its closed position, the Ultra-Violet sterilizers are activated.

### FIELD OF THE INVENTION

The present invention relates to a trash can, and more particularly, to a trash can with a lid which is equipped with Ultra-Violet sterilizers to kill germs and remove odors of the trash.

### BACKGROUND OF THE INVENTION

The conventional trash cans, especially for those trash cans with food waste organics such as kitchen scraps generally involve multiple types of bacteria, germs and odors. Although most of the trash cans have a lid to cover the top opening, the result cannot be satisfied. The conventional trash cans also bring health concerns to the area where the humidity and temperature are high and suitable for reproduction of the germs and bacteria.

There are some trash cans equipped with sterilizers such as the Ultra-Violet sterilizer or photocatalysis to kill germs and bacteria. However, when the lid is opened, the Ultra-Violet rays or photocatalysis are exposed and which may cause bad affect to people.

The present invention intends to provide a trash can which has a lid to seal the top opening of the can and Ultra-Violet sterilizers are connected to the underside of the lid. The operation of the Ultra-Violet sterilizers is controlled by a sensor on top of the can and the sensor detects the position of the lid to activate or de-activate the Ultra-Violet sterilizers.

### SUMMARY OF THE INVENTION

The present invention relates to a trash can and comprises a can and a lid which is pivotably connected to the open top of the can. The lid has multiple Ultra-Violet sterilizers connected to the underside of the lid. A sensor is connected on the top surface of the can. A control unit is connected to the can and has a power supply which is electrically connected to the Ultra-Violet sterilizers and the sensor. The Ultra-Violet sterilizers are activated when the sensor detects that the lid is in closed position.

Preferably, the sensor is a push-type sensor or an infra-red sensor.

Preferably, the power supply is a battery, a rechargeable member or an exterior power supply.

The primary object of the present invention is to provide a trash can having Ultra-Violet sterilizers connected to the underside of the lid so that when the lid is in closed position, the Ultra-Violet sterilizers are activated, and the Ultra-Violet sterilizers are de-activated when the lid is opened.

The control unit is able to set an operation time for the Ultra-Violet sterilizers which are in function during the period that the users set.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows the connection of the parts of the trash can of the present invention;

FIG. 2 is a perspective view to show the trash can of the present invention, wherein the lid is in opened position, and

FIG. 3 is a perspective view to show the trash can of the present invention, wherein the lid is in closed position.

### DETAILED DESCRIPTION OF THE PREFERRED EMBODIMENT

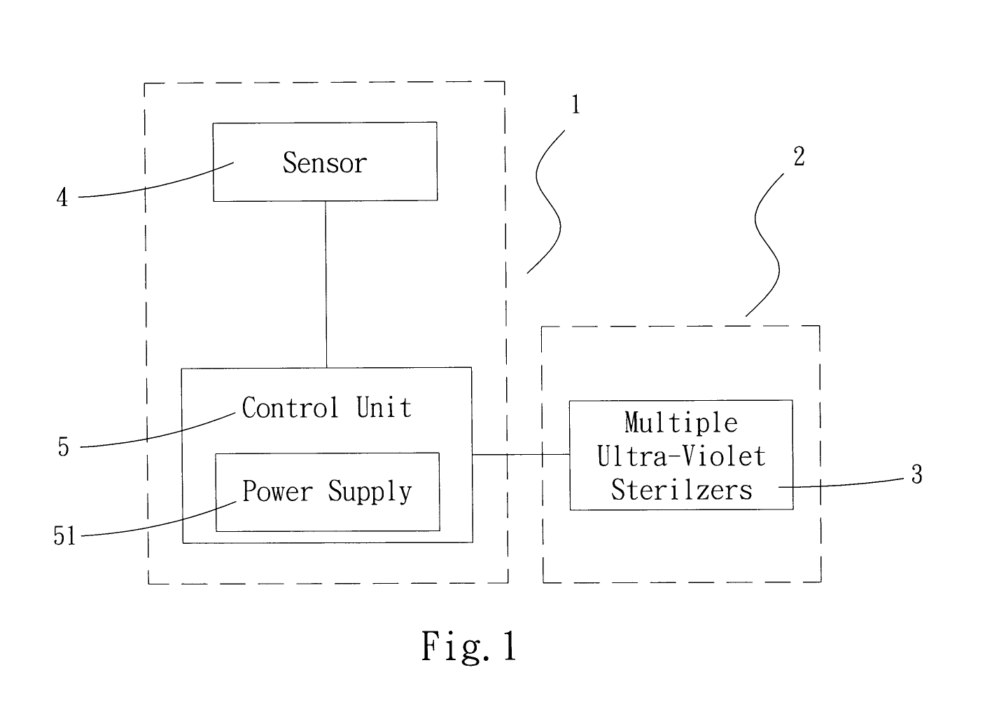

Referring to FIGS. 1 to 3, the trash can of the present invention comprises a can 1, a lid 2, multiple Ultra-Violet sterilizers 3, a sensor 4, a control unit 5 and a power supply 51.

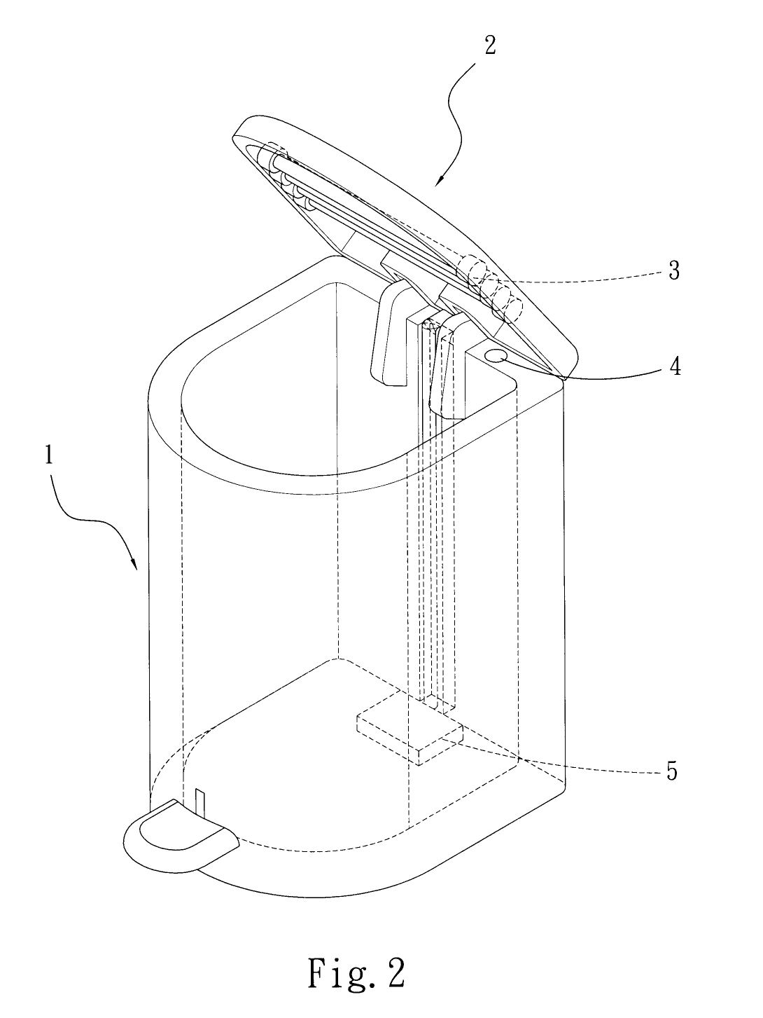

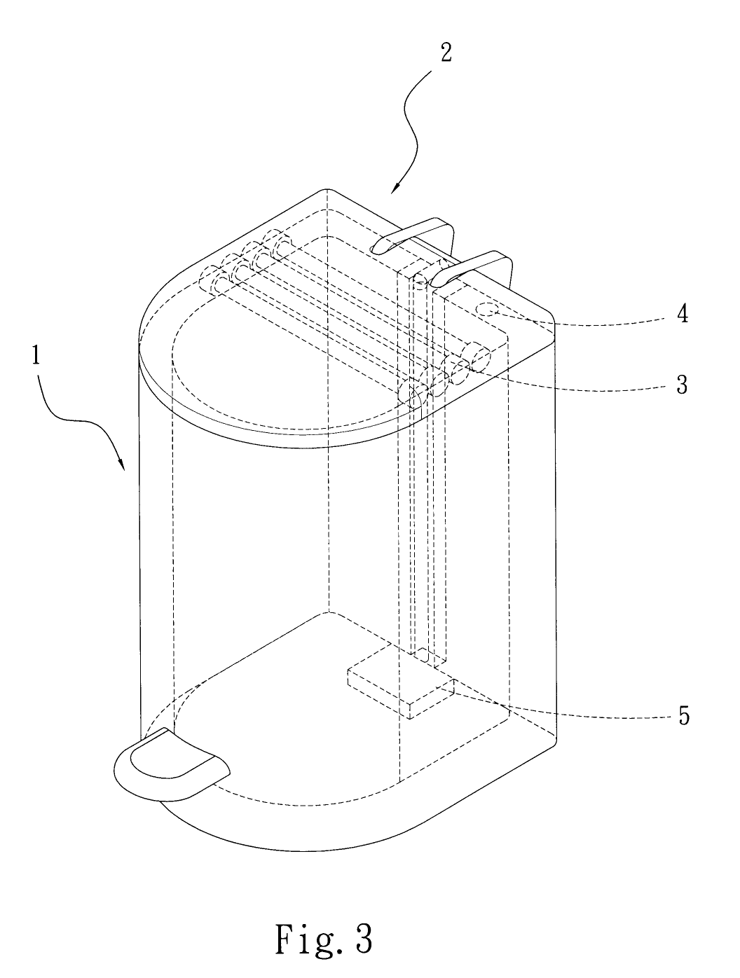

The lid 2 is pivotably connected to the open top of the can 1, and the lid 2 can be opened by manual, by sensors, or by operation of a pedal. As disclosed in FIGS. 2 and 3, the lid 2 is opened by the operation of a pedal. Multiple Ultra-Violet sterilizers 3 are connected to the underside of the lid 2 and the Ultra-Violet radiation is effective in controlling bacteria, yeast, mold and pathogens, and further controlling the generation of odors. The Ultra-Violet sterilizers 3 can be Ultra-Violet tubes or Ultra-Violet Light Emitting Diodes.

The sensor 4 is connected on the top surface of the can 1 and covered when the lid 2 is closed so that the sensor 4 can detect the position of the lid 2. The sensor 4 is located close to the pivotal portion of the lid 2 or any position on the can 1. The sensor 4, in this embodiment, can be a push-type sensor or an infra-red sensor.

The control unit 5 is connected to the can 1 and has a power supply 51. The control unit 5 is electrically connected to the Ultra-Violet sterilizers 3 and the sensor 4. When the sensor 4 detects that the lid 2 is in closed position, Ultra-Violet sterilizers 3 are activated. When the sensor 4 detects that the lid 2 is in opened position, Ultra-Violet sterilizers 3 are de-activated. The control unit 5 is able to set an operation time for the Ultra-Violet sterilizers 3 which are in function during the period that the users set.

The power supply 51 provides electric power to the Ultra-Violet sterilizers 3, the sensor 4, and the control unit 5, and can be a battery, a rechargeable member or an exterior power supply.

As shown in FIG. 2, when the user steps on the pedal, the lid 2 is opened and the sensor 4 detects that the lid 2 is opened, the control unit 5 de-activates the Ultra-Violet sterilizers 3 to prevent the Ultra-Violet radiation from being exposed. As shown in FIG. 3, when the lid 2 is closed, the sensor 4 detects the closed lid 2, the Ultra-Violet sterilizers 3 are activated. The operation time of the Ultra-Violet sterilizers 3 can be set to save energy as needed.

The trash can effectively control the bacteria and germs in the can and especially effective to the wastes generated in kitchens, washrooms and clinics. The Ultra-Violet radiation is effective in controlling bacteria, yeast, mold and pathogens, and further controlling the generation of odors. The Ultra-Violet sterilizers 3 are automatically activated when the lid 2 is closed and de-activated when the lid 2 is opened. The present invention can be easily installed to any trash can with a lid and the operation time of the Ultra-Violet sterilizers 3 can be controlled.

While we have shown and described the embodiment in accordance with the present invention, it should be clear to those skilled in the art that further embodiments may be made without departing from the scope of the present invention.

What is claimed is:

1. A trash can comprising:
a can and a lid pivotably connected to an open top of the can, multiple Ultra-Violet sterilizers connected to an underside of the lid;
a sensor connected on a top surface of the can;
a control unit connected to the can and having a power supply, the control unit electrically connected to the Ultra-Violet sterilizers and the sensor, when the sensor detects that the lid is in closed position, Ultra-Violet sterilizers are activated.

2. The trash can as claimed in claim 1, wherein the sensor is located close to a pivotal portion of the lid.

3. The trash can as claimed in claim 1, wherein the sensor is a push-type sensor or an infra-red sensor.

4. The trash can as claimed in claim 1, wherein the power supply is a battery, a rechargeable member or an exterior power supply.
